(19) United States
(12) Patent Application Publication
(10) Pub. No.: US 2013/0047644 A1
(43) Pub. Date: Feb. 28, 2013

(54) Use of Vetiver material to provide shades, cooling, humidification and air filteration for residential, commercial, indoor and outdoor facilities.

(71) Applicants: Viken Shah, Carol Stream, IL (US); Manish Shah, Naperville, IL (US); Pinku Shah, Carol Stream, IL (US)

(72) Inventors: Viken Shah, Carol Stream, IL (US); Manish Shah, Naperville, IL (US); Pinku Shah, Carol Stream, IL (US)

(21) Appl. No.: 13/214,700

(22) Filed: Aug. 22, 2011

(51) Int. Cl. F28D 5/00; A61L 9/04; A47H 23/00; F24F 6/00; E06B 9/386; F28D 15/00; B01D 46/00

(52) U.S. Cl. 62/121; 165/104.34; 62/314; 239/6; 95/273; 261/128; 160/236

(57) ABSTRACT

This patent covers the any use of vetiver roots material to provide shade, cooling and humidifying effect to any indoor or outdoor area. The use of vetiver root material could be in form of a mat, carpet or rug, however, not limited to them in their use to provide cooling and humidifying effect. A wet or dry mat using vetiver roots is placed in path of any natural or forced air to cool the air that is passing through. The air may be naturally flowing or forced via fan through the mat to provide cooling effect as well as cleaning effect. The patent also covers the use of water with the vetiver mat to provide additional cooling and humidity. The solution significantly reduces energy consumption.

### FIELD OF INVENTION

The present invention provides an environmentally sustainable cooling and humidifying solutions by using vetiver root material in form of mat, carpet or rug. Vetiver is a type of grass that is grown mostly in Asia. The roots are known for their eco conservation applications such as water-run off prevention, land slide prevention, river bed stabilization.

The present invention uses the same vetiver roots material to make a mat/carpet/rug by binding them together. The invention may be used to build outdoor/indoor cooling centers, humidifiers, greenhouse filters. The application mostly addresses either all or one of air cooling, air humidifying, providing shades, purifying air and provide natural aroma.

### BACK GROUND OF THE INVENTION

### Problem Statements

Traditional method of cooling and humidifying involves electrically powered air-conditions, which consumes large amount of electricity, emit significant heat and may require coolant solution which consists of strong chemicals. These chemicals are harmful to environment during production as well as consumption. The filters made for air-conditions, humidifiers and greenhouses use non-environmentally friendly material such as papers, metals and plastics or the combination of. These filters may be recycled but are not eco-friendly. They cannot be easily discarded and have significant half-life.

The manufacturing cost of making these traditional cooling solutions, humidifiers and filters is the long-term environmental impact, high cost of research/development and other resources. Cost of ownership is also very high, since the filter has to be changed and then discarded; air conditioning and humidifiers have to be maintained or fixed every few years. They stay in landfill for very long time and release toxic into the environment.

In summary the present cooling solutions consume large amount of energy, environmentally unfriendly, and costly. There is lot of room for improvement in all three aspects of the solution.

### Resolution

The answer lies in nature. Natural Vetiver roots possess some of the characteristics required to address problem statement above. Our invention uses Vetiver roots to build solutions that resolve problem statements shown above.

Vetiver Solutions proposes to use Vetiver roots as a natural cooling solution. One of the natural characteristics of vetiver root is its ability to naturally cool air passing through it. Our idea is to use this characteristic to build forced cooling solutions. The forced cooling solution uses vetiver roots mat, electric fan and an enclosure which can be mounted in any surrounding. The solution also incorporates a way to supply water to enhance cooling effect.

Vetiver Solutions also proposes to use Vetiver roots mat as a natural cooling solutions. The invention is to deploy vetiver mats as a shade in an outdoor environment or on windows. If the setup allows, Vetiver mats may be supplied with water to enhance cooling. The natural air passing through the mat is cooled and provides significant relief.

Vetiver Solutions also proposes to use Vetiver roots mat as an air filter or humidifier filter. This filter can be used for example in green houses or humidifying devices. The claim is that when air passes through the Vetiver mat is provides natural humidification with cooling and cleans the air particles.

1) Passing natural or forced air through vetiver root material (Mats, Carpets, Rugs, etc) to cool air
a. The air may naturally pass through mat
b. The air may be forced through the mat using manual or electrical fans/blower

2) Producing humidifying, cooling and shade effect after passing of natural of forced air through vetiver root material (Mat, Rug, etc) after water is applied the vetiver root material.
a. The water may be supplied from the top or may be sprayed
b. The vetiver root material may be soaked prior to passing air through it
c. The air may naturally pass through vetiver root material
d. The air may be forced through the vetiver root material using manual or electrical fans/blower
e. Any mechanism in which water passes through the vetiver root material is collected and recycled back in to the material using a motorized or mechanical device or pump

3) The use of vetiver roots material in a forced cooling system consisting of fan or fans that forces air through material in presence or absence of water supply.

4) The use of ability of vetiver root material to hold moisture to produce humidity

5) Use of vetiver root material to clean, purify and humidify air
a. A sample application (not limited to) is a green house filter where vetiver root material may be installed on one side where air comes in from.
i. The air gets purified
ii. The air gets cooled
iii. The air gets humidified
b. Another application (not limited to) is cooling centers.
i. Vetiver Material mat is hung on one or many sides of cooling centers to provide shade as well as cool and humid air
ii. The water may or may not be supplied to the vetiver root material mat

6) Any deployment of vetiver root material to provide natural shade from sun

7) The application of Vetiver roots material sed to make blinds and/or shades which can be deployed inside or around a home or any building to provide shade from sun

8) Use of vetiver root material to provide pleasant natural aroma to its indoor and outdoor surrounding
